(19) United States
(12) Patent Application Publication
(10) Pub. No.: US 2010/0226962 A1
(43) Pub. Date: Sep. 9, 2010

(54) PERI-CORNEAL DRUG DELIVERY DEVICE

(76) Inventors: Theron R. Rodstrom, Cransfill Gap, TX (US); David A. Marsh, Fort Worth, TX (US); Alan L. Weiner, Arlington, TX (US); Larry Smith, II, Glen Rose, TX (US); Martin B. Wax, Westlake, TX (US)

Correspondence Address: ALCON, IP LEGAL, TB4-8, 6201 SOUTH FREEWAY, FORT WORTH, TX, 76134, US

(21) Appl. No.: 12/712,622

(22) Filed: Feb. 25, 2010

Related U.S. Application Data
(60) Provisional application No. 61/157,010, filed on Mar. 3, 2009.

(51) Int. Cl. A61F 2/14; A61P 27/02

(52) U.S. Cl. 424/427

(57) ABSTRACT

The present invention is directed to an ophthalmic peri-corneal drug delivery device. The device includes a core of matrix material and therapeutic agent and a coating over the core. One or more opening[s] extend through the coating to provide for release of the drug to the eye. Moreover, the device is designed to lay atop the external surface of the eye.

This application claims priority under 35 U.S.C. §119 to U.S. Provisional Patent Application No. 61/157,010, filed Mar. 3, 2009, the entire contents of which are incorporated herein by reference.

### TECHNICAL FIELD OF THE INVENTION

The present invention relates generally to the field of ocular devices, pharmaceutics, and methods of drug delivery to the eye. More particularly, it concerns peri-corneal ocular devices for the sustained delivery of a therapeutic compound to the eye

### BACKGROUND OF THE INVENTION

The pharmaceutical industry has developed a variety of techniques for delivering ophthalmic compositions, particularly those that include therapeutic agents, to the eye. Typical ophthalmic drug delivery techniques include topical application of ophthalmic compositions to the eye (e.g., by drops directly onto the eye) and intravitreal injections, which involve delivery of ophthalmic compositions to the vitreous of the eye with a needle (e.g., a syringe). Both of these techniques have drawbacks. One particular drawback common to both of these techniques is the frequency with which the applications of the ophthalmic compositions must occur to best treat ophthalmic maladies such as glaucoma, age related macular degeneration (AMD) and others. Patients often forget or otherwise fail to administer drops to their eyes and patients can miss doctor appointments and fail to receive their needed injections.

In view of these drawbacks, the pharmaceutical industry has dedicated significant resources to the development of drug delivery devices that provide for sustained delivery of ophthalmic compositions and/or therapeutic agents to the eye. Such devices are often capable of providing a continuous dose or intermittent doses of therapeutic agent to the eye over extended periods of time.

Various ocular drug delivery implants have been described in an effort to improve and prolong drug delivery. For example, U.S. Pat. No. 3,949,750 discloses a punctal plug made of a tissue-tolerable, readily sterilizable material, such as Teflon, HEMA, hydrophilic polymer, methyl methacrylate, silicone, stainless steel or other inert metal material. It is stated that the punctal plug may be impregnated with ophthalmic medication or that the punctal plug may contain a reservoir of the ophthalmic drug.

U.S. Pat. No. 5,053,030 discloses an intracanalicular implant that can be used as a carrier or medium for distributing medications throughout the body. U.S. Pat. No. 5,469,867 discloses a method of blocking a channel, such as the lacrimal canaliculus by injecting a heated flowable polymer into the channel and allowing it to cool and solidify. The polymer may be combined with a biologically active substance that could leach out of the solid occluder once it has formed in the channel.

WO 99/37260 discloses a punctal plug made of a moisture absorbing material, which is not soluble in water, such as a modified HEMA. It is also disclosed that an inflammation inhibitor, such as heparin, may be added to the material from which the punctal plug is made.

U.S. Pat. No. 6,196,993 discloses a punctal plug containing glaucoma medication. The medication is contained in a reservoir within the plug. The reservoir is in fluid communication with a pore through which the medication is released onto the eye.

U.S. Pat. No. 4,592,752 discloses a corneal drug delivery device. The device is substantially the size and curvature of the cornea upon which it is placed and it includes an aperture substantially the size and shape of the pupil of the eye.

U.S. Patent Application No. 2008/0181930 discloses a drug delivery device having a body that includes a matrix of a therapeutic agent and another material such as silicon. The body is coated with a material such as parylene and one or more pores extend from the outer surface of the coating to the outer surface of the body to allow for release of therapeutic agent.

Each of these devices can provide for some degree of sustained delivery of an ophthalmic composition. However, these devices, as well as other conventional devices, typically suffer from one or more drawbacks. As one example, many conventional devices require that they be applied through an invasive surgical procedure. As another example, many conventional devices have difficulty delivering desired amounts of therapeutic agent for desired amounts of time. As yet another example, many devices have difficulty maintaining their desired location relative to the eye and can be lost or undesirably moved. As still another example, many conventional devices can cause discomfort. Thus, there is a need for an ophthalmic drug delivery device that can overcome one, two or more of these drawbacks.

### SUMMARY OF THE INVENTION

The present invention is directed to an ophthalmic pericorneal drug delivery device. The device includes an annular body formed of a core and a coating disposed over the core. The annular body is sized and shaped such that the body extends substantially entirely about a cornea of a human eye when the annular body is disposed upon the human eye. The core is formed of a polymeric matrix material and a therapeutic agent is dispersed within the polymeric matrix material. The polymeric matrix material is permeable to the therapeutic agent. Preferably, the coating substantially entirely surrounds the core. Openings extend through the coating to the core for allowing the therapeutic agent to be released from the core out of the device and to the eye.

The present invention is also directed to a method of treating an ophthalmic disease. Accordingly, the device can be disposed upon the eye to gradually release the therapeutic agent to the eye over an extended time period. In a preferred embodiment, the device is disposed and maintained upon the eye without the use of any mechanical fastening elements that extend into and/or attach to the eyeball.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is a perspective view of an exemplary peri-corneal drug delivery device in accordance with an aspect of the present invention.

FIG. 2 is a perspective view of the device of FIG. 1 shown as applied to an eye.

FIG. 3 is a perspective cut-away sectional view of the device of FIG. 1.

FIG. 4 is a perspective view of another exemplary peri-corneal drug delivery device in accordance with an aspect of the present invention.

FIG. 5 is a perspective view of another exemplary peri-corneal drug delivery device in accordance with an aspect of the present invention.

FIG. 6 is a perspective view of the exemplary peri-corneal drug delivery device of FIG. 1 further including haptics in accordance with an aspect of the present invention.

FIG. 7 is a perspective view of the exemplary peri-corneal drug delivery device of FIG. 4 further including haptics in accordance with an aspect of the present invention.

FIG. 8 is a perspective view of the exemplary peri-corneal drug delivery device of FIG. 5 further including haptics in accordance with an aspect of the present invention.

### DETAILED DESCRIPTION OF THE INVENTION

The present invention is predicated upon the provision of a peri-corneal drug delivery device. The device is configured to be disposed upon the conjunctiva and, preferably, substantially extends about and/or substantially surrounds the cornea. The device includes a core substantially entirely surrounded and enclosed by a coating. The core is formed of a matrix material having therapeutic agent dispersed through the matrix material. The core can advantageously act as a reservoir for therapeutic agent. The matrix material is typically permeable to the therapeutic agent while the coating material is at least less permeable and preferably substantially impermeable to the therapeutic agent. As such, one or more openings typically extend through the coating to the core for allowing therapeutic agent to be released from the core to an eye, typically to the eye of human. In this manner, a substantial amount of control can be gained relative to the amount of therapeutic agent that is released from the device.

As used herein, “substantially impermeable” as it applies to the coating and the therapeutic agent means that less than 5% and more typically less than 2% of the therapeutic agent released from the drug delivery device is released by virtue of permeation through the coating.

The matrix material of the drug delivery device is preferably a polymeric material and more preferably a hydrophobic flexible polymer, which may be, for example, a silicone, a polyacrylate; polyurethane, or a combination of two or more of these polymers. When the material is silicone, the silicone can be any unrestricted silicone suitable for injection, compression, or transfer molding. Non-limiting examples of commercially available, unrestricted silicones that may be used in making the devices of the present invention include MED-4870, MED-4830, MED-4840, MED-4850, MED-4860, or MED-4880 (NuSil Technology LLC). Non-limiting examples of polyacrylates include polymers of 2-hydroxyethylmethacrylate (HEMA), methacrylic acid (MA), methyl methacrylate (MMA).

The therapeutic agent (e.g., ophthalmic drug) dispersed through the matrix may be any therapeutic agent, so long as the therapeutic agent is dispersible and/or miscible in the matrix material (e.g., a silicone matrix). The dispersion may be a solid, a semi-solid or a liquid. In a preferred embodiment, the therapeutic agent is formed of solid particles or is a liquid such as an oil. In particular embodiments, the therapeutic compound is a compound that can be applied for the treatment of an ophthalmic disorder. For example, the therapeutic compound may be a glaucoma medication, an antimicrobial medication, an anti-inflammatory medication, or a dry-eye syndrome medication, or a therapeutic compound that can be applied in the treatment of diabetic retinopathy or age-related macular degeneration.

It is typically desirable to use higher amounts of particle type therapeutic agents relative to fluid (e.g., oil) type therapeutic agents since it can be more difficult for the particle type agents to permeate out of the matrix. In particular for particle agents, fluid often needs to permeate into the matrix to dissolve the agent and allow the agent to permeate out of the matrix. For particle type agents, it is typically desirable for the drug to be at least about 20% or 30% by weight of the core.

Ophthalmic drugs, such as prostaglandins, triamcinolone, 15-HETE (Icomucret), anti-inflammatories (non-steroidal anti-inflammatory drugs (NSAIDs)) receptor tyrosine kinase inhibitors (RTKi), timolol maleate, fluoroquinolones (e.g., moxifloxacin) and rimexolone, are well suited for delivery with the devices of the present invention. The prostaglandin may be a natural or a synthetic prostaglandin. Non-limiting examples of prostaglandins include cloprostenol, fluprostenol, latanoprost, travoprost, and unoprostone.

In preferred embodiments of the invention, the concentration of the therapeutic agent in the core is at least 0.1%, more typically at least 0.5% and possibly at least 1.0% by weight. In such embodiments, the concentration of the therapeutic agent in the core is typically no greater than 50%, more typically no greater than 30% and even more typically no greater than 20% by weight. Of course, unless otherwise specifically stated, higher and lower concentrations may be employed depending upon the particular therapeutic agent.

It is also contemplated that surface active agent (e.g., surfactant) can be included in the polymer matrix. The therapeutic agent[s] may be combined with the surface active agent (e.g., within an aqueous solution) and dispersed within the matrix or the surface active agent and the therapeutic agent may be separately dispersed within the polymer matrix. Advantageously, the surface active agent can assist in increasing the rate at which the therapeutic agent is released from the matrix and, ultimately, from the device. Surface active agents (e.g., surfactants) suitable for the present invention include, without limitation, hydrophilic surfactants, lipophilic surfactants, ionic surfactants, non-ionic surfactants, polymeric surfactant, any combination thereof or any surfactant that falls under two or more of these categories. Difunctional block copolymer surfactants terminating in hydroxyl groups are one example of a class of desirable surfactant. Examples of such surfactants are commercially sold under the tradenames PLURONIC L64, PLURONIC L121 and PLURONIC F68, which are commercially available from BASF Corporation, 3000 Continental Drive-North, Mount Olive, N.J. 07828-1234. Other desirable surfactants include, without limitation, polysorbates (e.g., polysorbate 80), hydrogenated castor oils (e.g., polyoxyl-40 hydrogenated castor oil), sorbitan surfactants (e.g., Sorbitan oleate; Sorbitan (Z)-mono-9-octadecenoate), polyethylene ethers (e.g., Polyethylene (100) Stearyl Ether), any combinations thereof or the like.

When included, the surface active agent (e.g., surfactant) will typically be at least about 0.01 w/w %, more typically at least about 0.05 w/w % and even more typically at least about 0.1 w/w % of the core or any aqueous therapeutic agent containing solution in the core. The surface active agent will typically be no greater than about 5 w/w %, more typically no greater than about 2 w/w % and even more typically no greater than about 0.05 w/w % of the core or any aqueous therapeutic containing solution in the core.

The coating located on the outer surface of the body of the device preferably comprises a non-biodegradable polymer different from the matrix material. The polymer that forms the coating is substantially or entirely impermeable to the therapeutic compound, or is at least substantially less permeable to the therapeutic compound than the therapeutic compound is to the matrix material. Examples of potential coating materials include, without limitation, ethylene vinyl acetate (EVA), polyimide, polytetrafluoroethylene (PTFE), combination thereof or the like. In a highly preferred embodiment, the coating is formed of parylene. The coating typically has a thickness of at least about 0.5 nanometers (nm), more typically at least about 100 nm and even more typically at least about 1 micrometer (μm) and even possibly at least about 5 μm. The thickness of the coating is also typically no greater than about 50 μm, more typically no greater than about 20 μl, still more typically no greater than about 15 μm and even possibly no greater than about 10 μm.

One or more openings are etched or otherwise formed in the coating to permit the release of the therapeutic compound from the matrix. The opening size and/or the number of openings may be adjusted to achieve the desired release rate for the particular therapeutic agent in the matrix. In certain embodiments of the invention, the opening has a diameter, as measured at the outer surface of the coating or as measured at the interface of the coating and the core, of between about 1 μm to about 100 μm, about 1 μm to about 50 μm, or about 5 μm to about 50 μm. In certain embodiments, the number of openings is between 1 to about 100,000; 1 to about 20,000; 1 to about 10,000; 1 to about 2,000; 1 to about 1,000; 1 to about 100; 1 to about 50; 1 to about 10; 1 to about 8; 1 to about 5; about 5 to about 100; about 5 to about 10; about 10 to about 100,000; about 10 to about 10,000; about 10 to about 2,000; about 10 to about 1,000; about 10 to about 100; about 100 to about 100,000; about 100 to about 10,000; about 100 to about 2,000; or about 100 to about 1,000.

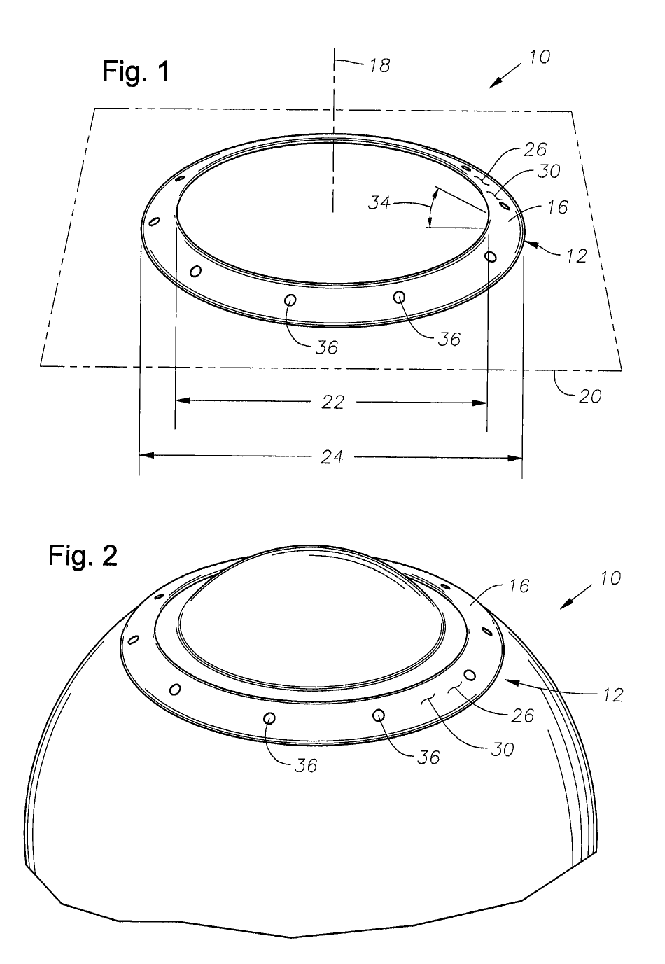

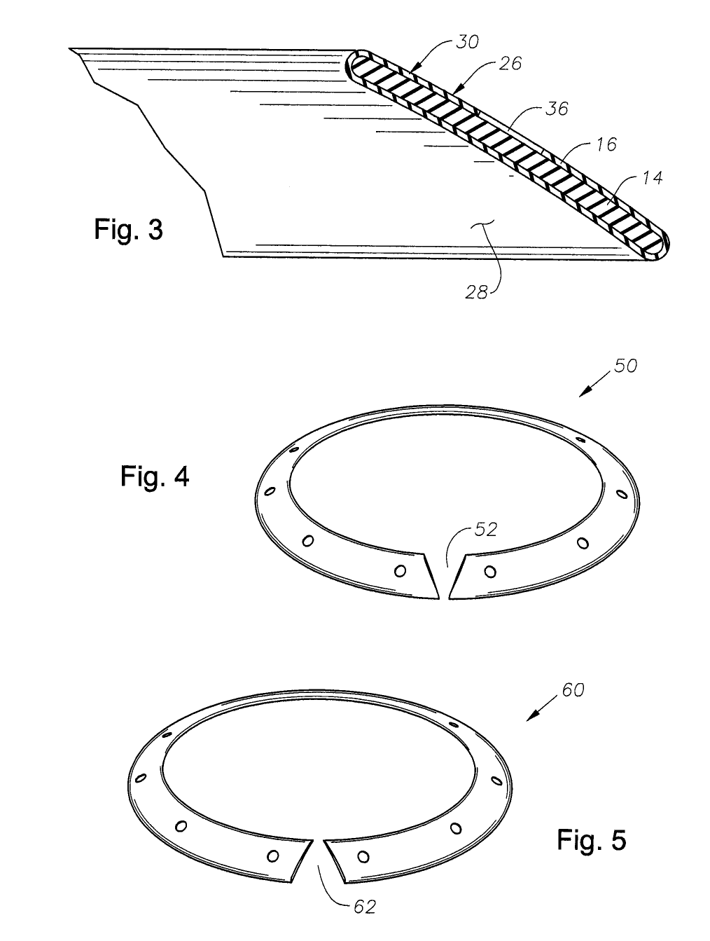

With reference to FIGS. 1 through 3, there is illustrated an exemplary drug delivery device 10 according to an aspect of the present invention. The device 10 includes an annular portion 12 that includes a core 14 and a coating 16 disposed upon the core 14. The exemplary device 10, as illustrated, is annular about a central axis 18 and lies in a plane 20 that is perpendicular to that axis. In the embodiment shown, the device 10, including the core 14 and the coating 16, is in a continuous ring or band having an inner diameter 22 and an outer diameter 24. Moreover, the illustrated device 10, including the core 14 and the coating 16, is substantially or entirely symmetrical about the central axis 18. The inner diameter is typically configured to be directly adjacent the cornea upon application of the device to the eye. A portion of the device may reside upon the outer periphery of the cornea, but this is typically not desired. The inner diameter of the device is typically at least 0.3 centimeter (cm), more typically at least 0.6 cm and even more possibly at least 0.9 cm and is typically no greater than 1.5, more typically no greater than 1.3 cm and even more typically no greater than 1.1 cm. The outer diameter is typically at least 0.6 cm, more typically at least 1.1 cm and even more possibly at least 1.3 cm and is typically no greater than 2.2, more typically no greater than 1.9 cm and even possibly no greater than 1.7 cm.

The device 10 generally has an outer surface 26. That outer surface 26 includes a first surface 28, which is a contacting surface that contacts the conjunctiva of the eye when the device 10 is placed atop the conjunctiva. The device 10, and particularly the outer surface 26, also includes a second surface 30 that is opposite the first surface 28. The second surface 30 is an outwardly facing surface that faces away from the conjunctiva upon placement of the device 10 thereon. The first surface 28 can be flat or slightly concave. The second surface 30 can be flat or slight convex. Both the first surface 28 and the second surface 30 are disposed as an angle 34 relative to the plane 20 in which the device 10 lies. That angle 34 may be different for different portions of the surface[s] 26, 28, but is typically at least about 3°, more typically at least about 10° and even possibly at least about 20° and is also typically no greater than about 60°, more typically no greater than about 45° and even possibly no greater than about 30°.

The device 10 of FIG. 1 includes one or more openings 36 extending from the outer surface 26, through the coating 16 and to the core 14. According to certain aspects of the present invention, the opening[s] are located only on the surface of the device that contacts the conjunctiva of the eye. Alternatively, the opening[s] can be located only on the surface facing away from the conjunctiva of the eye. As still another alternative, opening[s] may be located on both surfaces. Having openings extending through the surface facing away from the conjunctiva can be particularly desirable for delivery of anti-glaucoma or intraocular pressure lowering therapeutic agents such as a prostaglandin (e.g., coprostenol, fluprostenol, latanoprost, travoprost, and unoprostone). This allows the therapeutic agent to diffuse into the tear film and from the tear film through the cornea to the iris ciliary body. Having openings extending through the surface that contacts the conjunctiva can be particularly desirable for therapeutic agents that act at the posterior of the eye and can benefit from improved delivery to the vitreous. Such drugs can include anti-inflammatories, particularly NSAIDs such as nepafenac or diclofenac.

In certain aspects, the devices of the present invention deliver a therapeutically effective dose of the therapeutic compound to the subject for at least about 20 days, at least about 30 days, at least about 60 days, at least about 90 days, at least about 120 days, at least about 180 days, at least about 240 days, at least about 300 days, at least about 1 year, at least about 2 years, at least about 3 years, at least about 4 years, at least about 5 years, at least about 6 years, at least about 7 years, or at least about 8 years, or any range derivable therein. In particular embodiments, the devices of the present invention deliver a therapeutically effective dose of the therapeutic compound for at least 90 days.

Advantageously, the device of the present invention can provide desirable dosage amounts of therapeutic agent during the above referenced extended time periods. Generally, the device can typically deliver at least 0.01 m/day, more typically at least 0.1 μg/day and even more typically at least 0.6 μg/day of therapeutic agent. The device also typically delivers no greater than 1000 μg/day, more typically no greater than 400 μg/day and still more typically no greater than 150 μg/day of therapeutic agent. For higher potency drugs such as prostaglandins, the device is typically configured to deliver from about 0.4 μg/day to about 2.0 μg/day of therapeutic agent. For lower potency drugs, the device is typically configured to deliver from about 30 μg/day to about 120 μg/day of therapeutic agent.

In one embodiment, the present invention provides a method of manufacturing a drug-delivery device, comprising: (a) forming (e.g., molding) the matrix material with a therapeutic agent dispersed therein to form a core of the drug delivery device and (b) forming (e.g., by dip-coating, overmolding or the like) a coating over the core, the coating preferably substantially or entirely encapsulating the core; and (c) forming (e.g., drilling, molding or etching) one or more opening[s] in the coating, wherein the openings[s] extends from an outer surface of the coating at least to the surface of the core. Examples of molding or incorporating a therapeutic agent into a matrix material are in U.S. Patent Application Numbers 2008/0113027 and 2008/0181930, both of which are fully incorporated herein by reference for all purposes.

In certain aspects of the invention, the coating is deposited using vapor deposition. In particular embodiments, the opening[s] are etched using oxygen plasma etching or focused ion beam etching. In certain embodiments, the parylene coating is deposited at a thickness of between about 0.5 nanometers (nm) to about 100 micrometers (μm), about 100 nm to about 50 μm, or about 1 μM to about 10 μm. In certain aspects of the invention, the opening[s] are substantially circular. The number of openings and the size of the openings etched in the coating may be adjusted to achieve the desired release rate for the therapeutic agent.

In one embodiment, the present invention provides a method of treating an ocular disorder in a subject comprising: (a) forming a drug delivery device as described herein for the sustained release of therapeutic agent to the eye; and (b) disposing the device upon an external surface (e.g., surface of the conjunctiva) of the eye. The method can be specifically for treating glaucoma or ocular hypertension in a subject (e.g., a human) and the therapeutic agent can be, for example, a prostaglandin.

In some embodiments, biodegradable microspheres of the therapeutic agent are formed first and then incorporated into a the matrix material. Microspheres, microcapsules and nanospheres (collectively, “microspheres”) are generally accepted as spherical particles with diameters ranging from approximately 50 nm to 1000 micrometers. They are reservoir devices that come in a variety of different forms, including, but not limited to, porous, hollow, coated, or uncoated forms with a pharmaceutically active agent either incorporated into or encapsulated by polymeric material via numerous known methods. Such known methods include, but are not limited to, spray drying, spinning disk and emulsification methods. Microspheres may be formed from a myriad of polymeric materials selected from, but not limited to, polylactic acids, polyglycolic acids, polylactic-glycolic acids, poly caprolactones, triglycerides, polyethylene glycols, poly orthoesters, poly anhydrides, polyesters, cellulosics and combinations thereof. The amount of therapeutic agent incorporated or encapsulated in the microsphere is generally between 0.001% and about 50%. In this embodiment, preformed microspheres are incorporated into the drug-delivery device body such that the core comprises a matrix of a silicone and drug-loaded microspheres. The microsphere content incorporated into the drug-delivery device core is generally between 1% and 50%.

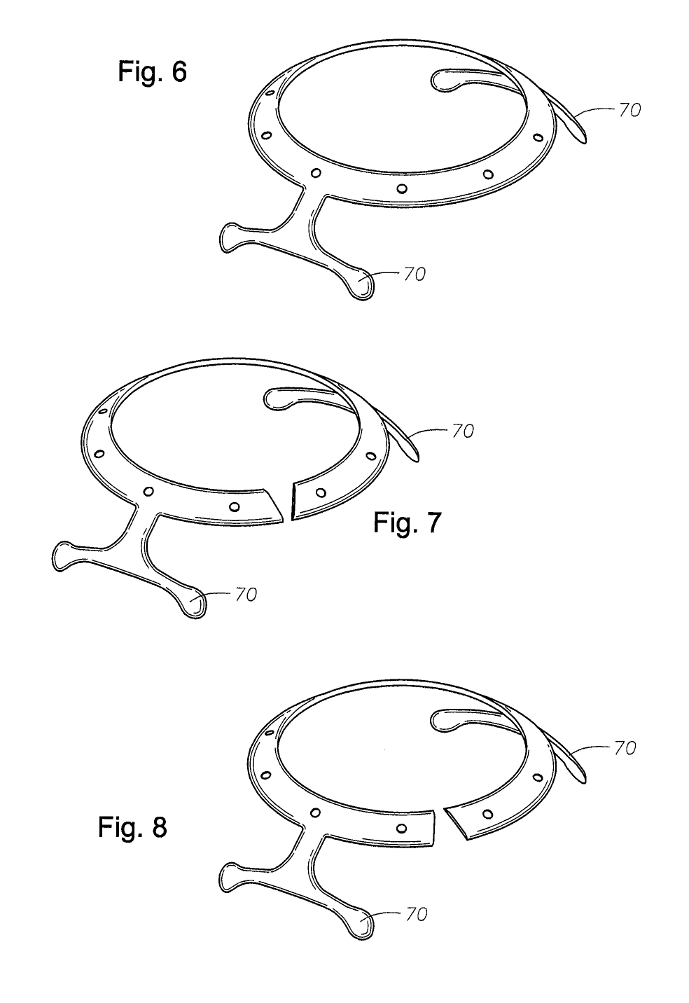

It is also contemplated that the drug delivery device of the present invention can have different forms and additional elements relative to the embodiments of FIGS. 1 through 3. With reference to FIG. 4, another exemplary drug delivery device 50 according to the present invention is illustrated. The device 50 is substantially identical to the device of FIG. 1 with the exception that the annular portion of the device 50 includes a gap 52 that constricts from the outer diameter to the inner diameter. It should be understood that the term “annular portion” as used herein refers to the portion of the device that forms or substantially forms a ring (i.e., forms at least 60% and more preferably at least 80% of a ring that is designed to extend about the cornea). With reference to FIG. 5, another exemplary drug delivery device 60 according to the present invention is illustrated. The device 60 is substantially identical to the device of FIG. 1 with the exception that the annular portion of the device 60 includes a gap 62 that constricts from the inner diameter to the outer diameter. With reference to FIGS. 5-6, the devices of FIGS. 1, 3 and 4 have been modified to include haptics 70 that extend outwardly from the annular portion of the device. Advantageously, these haptics can aid in maintaining the device in place relative to the eye.

Whichever design is used for the device, the device can be configured to have a relatively large external surface area, which allows the device to be maintained upon the conjunctiva more securely. In particular, capillary forces of the fluid upon the conjunctiva can aid in maintaining the device upon the eye. It should be noted that, for purposes of this invention, the fluid located upon the conjunctiva is considered to be part of the conjunctiva upon which the device can be located. The surface area of the contacting surface as determined inclusive of any and every portion (including haptics) of the device that contacts the conjunctiva is typically at least 50 (millimeters squared) mm², more typically at least 77 mm², even more typically at least 90 mm² and even possibly at least 110 mm² and the surface area of that portion is typically no greater than 320 mm², more typically no greater than 220 mm², even more possibly no greater than 170 mm² and even possibly no greater than 120 mm².

Advantageously, it may be the case that the device of the present invention can reside upon and be maintained upon the eye without needing any fastening elements such as stitches or other mechanical devices that extend into the eye (i.e., into the conjunctiva, cornea or any other portion of the eyeball). Such fastening devices typically must be surgically applied and avoidance of such surgical applications can be desirable in many circumstances.

The device of the present invention can, in certain embodiments, be relatively large and can include a relatively large core, which can delivery relatively large amounts of therapeutic agent over the extended time periods. The volume of the entire device of the present invention is typically at least 10 mm³, more typically at least 14 mm³, and even more typically at least 18 mm³ and the volume of the device is typically no greater than 100 mm³, more typically no greater than 50 mm³, and even possibly no greater than 30 mm³. The weight of the entire device of the present invention is typically at least 10 mg, more typically at least 14 mg, and even more typically at least 17 mg and the weight of the device is typically no greater than 1000 mg, more typically no greater than 100 mg, and even more possibly no greater than 30 mg.

The use of the term “or” in the claims is used to mean “and/or” unless explicitly indicated to refer to alternatives only or the alternatives are mutually exclusive.

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

In this document (including the claims), the terms “comprise” (and any form of comprise, such as “comprises” and “comprising”), “have” (and any form of have, such as “has” and “having”), and “include” (and any form of include, such as “includes” and “including”) are open-ended linking verbs.

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

Other embodiments of the present invention will be apparent to those skilled in the art from consideration of the present specification and practice of the present invention disclosed herein. It is intended that the present specification and examples be considered as exemplary only with a true scope and spirit of the invention being indicated by the following claims and equivalents thereof.

We claim:

1. An ophthalmic pericorneal drug delivery device, comprising;
an annular body formed of a core and a coating disposed over the core, the annular body is sized and shaped such that the body resides upon a conjunctiva of a human eye and extends substantially entirely about a cornea of the human eye when the annular body is disposed upon the human eye, wherein:
i. the core is formed of a polymeric matrix material and a therapeutic agent dispersed within the polymeric matrix material;
ii. the polymeric matrix material is permeable to the therapeutic agent;
iii. the coating substantially entirely surrounds the core; and
iv. openings extend through the coating to the core for allowing the therapeutic agent to be released from the core out of the device and to the eye.

2. A device as in claim 1 wherein the therapeutic agent is a prostaglandin.

3. A device as in claim 1 wherein the device, the annular body or both include a contact surface that is shaped and sized to correspond to and contact the conjunctiva of the human eye upon application of the device to the eye.

4. A device as in claim 3 wherein the contact surface of the device including all portions that contact the conjunctiva has a surface area that is at least 77 mm² and is no greater than 220 mm².

5. A device as in claim 1 wherein the device has a volume that is at least at least 14 mm³ and is no greater than 100 mm³.

6. A device as in claim 1 wherein one or more haptics extends outward from the annular body and such haptics assist in maintaining the desired location of the annular body upon the conjunctiva.

7. A device as in claim 1 wherein the device delivers a load of the therapeutic agent to the eye.

8. A device as in claim 1 wherein the annular body has an inner diameter that is at least 0.6 centimeters and no greater than 1.3 centimeters.

9. A device as in claim 1 wherein the device is configured to deliver a therapeutically effective amount of the therapeutic agent to the eye for an extended time period of at least 20 days.

10. A method of treating an ophthalmic disease comprising:
disposing a device as in claim 1 on the conjunctiva of the eye.

11. A method as in claim 10 wherein the device is disposed and maintained upon the eye without the use of any mechanical fastening elements that extend into the eyeball.

12. An ophthalmic pericorneal drug delivery device, comprising;
an annular body formed of a core and a coating disposed over the core, the annular body is sized and shaped such that the body resides upon a conjunctiva of a human eye and extends substantially entirely about a cornea of the human eye when the annular body is disposed upon the human eye, wherein:
i. the core is formed of a polymeric matrix material and a therapeutic agent dispersed within the polymeric matrix material;
ii. the polymeric matrix material is permeable to the therapeutic agent;
iii. the coating substantially entirely surrounds the core;
iv. openings extend through the coating to the core for allowing the therapeutic agent to be released from the core out of the device and to the eye;
v. the device, the annular body or both include a contact surface that is shaped and sized to correspond to and contact the conjunctiva of the human eye upon application of the device to the eye;
vi. the contact surface of the device including all portions that contact the conjunctiva has a surface area that is at least 77 mm² and is no greater than 220 mm²
vii. the annular body has an inner diameter that is at least 0.6 centimeters and no greater than 1.3 centimeters; and
viii. the device is configured to deliver a therapeutically effective amount of the therapeutic agent to the eye for an extended time period of at least 20 days.

13. A device as in claim 12 wherein the therapeutic agent is a prostaglandin.

14. A device as in claim 12 wherein the device has a volume that is at least at least 14 mm³ and is no greater than 100 mm³.

15. A device as in claim 12 wherein one or more haptics extends outward from the annular body and such haptics assist in maintaining the desired location of the annular body upon the conjunctiva.

16. A device as in claim 12 wherein the device delivers a load of the therapeutic agent to the eye.

17. A method of treating an ophthalmic disease comprising:
disposing a device as in claim 12 on the conjunctiva of the eye.

18. A method as in claim 17 wherein the device is disposed and maintained upon the eye without the use of any mechanical fastening elements that extend into the eyeball.
